(19) United States
(12) Patent Application Publication
(10) Pub. No.: US 2026/0209672 A1
(43) Pub. Date: Jul. 23, 2026

(54) BIOPROCESSING SYSTEM WITH AUTOMATIC FILTER SWITCHING AND ROTARY VALVE FOR USE IN A BIOPROCESSING SYSTEM

(71) Applicant: Repligen Corporation, Waltham, MA (US)

(72) Inventor: Rudolf Pavlik, Lake Forest, CA (US)

(73) Assignee: Repligen Corporation, Waltham, MA (US)

(21) Appl. No.: 19/135,878

(22) PCT Filed: Dec. 14, 2023
(86) PCT No.: PCT/US2023/083991

§ 371 Date: Jun. 5, 2025

Related U.S. Application Data
(60) Provisional application No. 63/433,556, filed on Dec. 19, 2022.

(51) Int. Cl. C12M 1/00; B01D 63/02; C12M 1/34; C12M 1/36

(52) U.S. Cl. CPC C12M 29/04; B01D 63/02; C12M 23/40; C12M 41/40; C12M 41/48; B01D 2313/18; B01D 2313/243; B01D 2313/60; B01D 2313/70

(57) ABSTRACT

A bioprocessing system includes a process vessel, a plurality of filter housings, a feed pump for moving fluid between the process vessel and the filtration assembly, and a retentate valve within a retentate line. The retentate valve includes a fluid passageway to selectively direct movement of the fluid to at least one of the plurality of filter housings. A permeate valve is provided in a permeate line between the filter housings and a permeate pump. A pressure sensor is provided in the permeate line, and a controller is coupled to the pressure sensor, the retentate valve, and the permeate valve. The controller actuates the retentate and permeate valves to direct flow between the process vessel one or more of the filter housings, and to direct permeate flow from the filter housings to the permeate pump based on information received from the flow sensor and the pressure sensor.

### CROSS-REFERENCE TO RELATED APPLICATIONS

This application is a non-provisional of pending U.S. Provisional Patent Application Ser. No. 63/433,556, filed Dec. 19, 2022, the entirety of which application is incorporated by reference herein.

### FIELD OF THE DISCLOSURE

The disclosure relates generally to filtration systems used in connection with pharmaceutical or bioprocess applications. More specifically, the disclosure relates to filtration systems that enable selection between one or more filters in a multi-filter bank used in pharmaceutical or bioprocess applications, while minimizing or eliminating the presence of holdup volumes.

### BACKGROUND

As will be appreciated by one of ordinary skill in the art, bioreactor systems or process filtration systems are known for cultivating cell cultures. Cultures of microbial, plant, or animal cells may be used to produce biological and chemical substances of significant commercial value. Generally speaking, a bioreactor or process filtration system may use a perfusion culture using a cell retention device to continuously replenish cell culture media, remove waste products, and harvest product, while retaining the cells within the bioreactor or process filtration system.

Biologics manufacturing processes have advanced through substantial process intensification. Both eukaryotic and microbial cell culture to produce recombinant proteins, virus-like particles (VLP), gene therapy particles, and vaccines now include cell growth techniques that can achieve 100e6 cells/ml or higher. This is achieved using cell retention devices that remove metabolic waste products and refresh the culture with additional nutrients. One of the most common means of cell retention is to perfuse a bioreactor culture using hollow fiber filtration using alternating tangential flow (ATF).

Many commercial products are produced using biological processes. Pharmaceuticals, for example, are produced in commercial quantities using scaled-up reactors and other equipment. So-called biologics are drugs or other compounds that are produced or isolated from living entities such as cells or tissue. Biologics can be composed of proteins, nucleic acids, or complex combinations of these substances. They may even include living entities such as cells. In order to produce biologics on a commercial scale, sophisticated and expensive equipment is needed. In both pharmaceutical and biologics, for example, various processes need to occur before the final product is obtained. For example, in the case of biologics, cells may be grown in a growth chamber or the like and nutrients may need to be carefully modulated into the growth chamber. Waste products produced by cells may also have to be removed on a controlled basis from the fermentation chamber. As another example, biologic products produced by living cells or other organisms may need to be extracted and concentrated. This process may involve a variety of filtration and separation techniques.

Filtration is typically performed to separate, clarify, modify and/or concentrate a fluid solution, mixture or suspension. In the biotechnology and pharmaceutical industries, filtration is vital for the successful production, processing, and testing of new drugs, diagnostics and other biological products. For example, in the process of manufacturing biologicals, using animal or microbial cell culture, filtration is done for clarification, selective removal and concentration of certain constituents from the culture media or to modify the media prior to further processing. Filtration may also be used to enhance productivity by maintaining a culture in perfusion at high cell concentration.

When filters are used in a bioprocessing system, their permeability reduces over time. When the permeability of the filters reduces below a certain level, the filter media must be changed. Filter changes during filtration operation are typically complex and represent an interruption in the filtration process, along with associated loss of product and reduced productivity. Filter changes in sterile systems also introduce the potential for contamination of some or all of the system.

Further, existing filtration systems often suffer from the creation of larger than desired holdup volumes, and the presence of dead legs in the wetted fluid paths. Both issues can adversely impact product recovery, separation efficiency, fluid volumes required (cleaning, flushing, and processing), system cost and required floor space, and the ease of cleaning and sanitizing the system.

It is with respect to these issues that the present disclosure is provided.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

The disclosed system helps to mitigate problems associated with filter changes required during continuous filtration operations. The disclosed system also reduces or eliminates the formation of holdup volumes in dead legs of tubing, valves and the like that often form when filtration flow paths are changed, such as during filter change operations.

A bioprocessing system is disclosed, including a process vessel, a filtration assembly including a plurality of filter housings, a feed pump coupled to the plurality of filter housings for moving fluid between the process vessel and the filtration assembly, a retentate valve coupled within a retentate line between the process vessel and the plurality of filter housings, the retentate valve including a fluid passageway that is controlled to selectively direct movement of the fluid to at least one of the plurality of filter housings, a permeate valve coupled within a permeate line between the plurality of filter housings and a permeate pump, a pressure sensor disposed in the permeate line, and a controller coupled to the pressure sensor, the retentate valve, and the permeate valve. The controller can execute instructions for actuating the retentate valve and the permeate valve to direct flow between the process vessel and a selected one of the plurality of filter housings, and to direct permeate flow from the selected one of the plurality of filter housings to the permeate pump, based on information received from the pressure sensor.

In some embodiments the system further includes a pneumatic valve coupled between a source of air pressure and a plurality of pneumatically actuated pumps associated with respective ones of the plurality of filter housings. This is, of course, not critical, and different pumping arrangements (i.e., non-pneumatically activated) can be used.

In some embodiments a flow sensor is disposed in the retentate line for monitoring a flowrate of the feed to and from the process vessel.

In some embodiments the retentate valve is a rotary retentate valve having a rotor with a fluid passageway, the rotor being rotatable to orient the fluid passageway to fluidly connect an inlet of the rotary retentate valve with a selected outlet of the rotary retentate valve.

In some embodiments the rotary retentate valve comprises a plurality of outlets, and wherein each of the plurality of outlets is fluidly coupled to a respective one of the plurality of filter housings.

In some embodiments the fluid passageway comprises a plurality of fluid passageways, and the selected outlet comprises a plurality of selected outlets, and the rotor is rotatable to orient the plurality of fluid passageways to fluidly connect the inlet of the rotary retentate valve with the selected plurality of outlets.

In some embodiments the permeate valve is a rotary permeate valve having a rotor with a fluid passageway, the rotor being rotatable to orient the fluid passageway to fluidly connect an inlet of the rotary permeate valve with a selected outlet of the rotary permeate valve.

In some embodiments the inlet of the rotary permeate valve is fluidly coupled to the permeate line.

In some embodiments the retentate valve is a rotary retentate valve having a housing and a rotor disposed within the housing, the rotor including a cylindrical portion and a conical portion, the conical portion seated within a corresponding conical portion of the housing and disposed for rotational movement about a rotational axis therein.

In some embodiments the retentate valve is a rotary retentate valve having a housing and a cylindrical rotor disposed within the housing, the cylindrical rotor including a plurality of fluid passageways for selectively fluidly coupling an inlet of the rotary retentate valve to a selected one of a plurality of outlets of the rotary retentate valve.

In some embodiments the rotary retentate valve includes an extension disposed through the housing, the extension coupled to an actuator controllable by the controller to change a rotational position of the rotor to orient at least one of a plurality of fluid passageways in the rotor to couple an inlet of the rotary retentate valve to a desired one of a plurality of outlets of the rotary retentate valve, wherein each of plurality of outlets is fluidly coupled to a respective one of the plurality of filter housings.

A rotary valve for use in a bioprocessing system includes a housing comprising a housing body and a housing cap, the housing body comprising a plurality of fluid outlets, and a rotor disposed within the housing body and having a fluid passageway, the rotor being rotatable within the housing body to fluidly connect a fluid inlet to a selected one of said plurality of fluid outlets.

In some embodiments the rotor further includes an extension member disposed through the housing and couplable to an actuator for rotating the rotor.

In some embodiments the fluid passageway comprises a plurality of fluid passageways.

In some embodiments the plurality of fluid passageways comprise three parallel fluid passageways.

In some embodiments the plurality of fluid passageways each includes a bend having a geometry configured to facilitate laminar flow through the valve.

In some embodiments the inlet is disposed in the housing cap, the inlet being fluidly coupled to an inlet portion of the fluid passageway.

In some embodiments the inlet is oriented to direct flow into the fluid passageway along a rotational axis of the rotor.

In some embodiments the inlet is disposed in the housing body and is oriented orthogonal to a rotational axis of the rotor.

In some embodiments the fluid passageway comprises a plurality of fluid passageways configured such that the rotor is rotatable to a first predetermined position in which the inlet is fluidly coupled to a first selected pair of fluid outlets of the plurality of fluid outlets.

In some embodiments the rotor is rotatable to a second predetermined position in which the inlet is fluidly coupled to a second selected pair of fluid outlets of the plurality of fluid outlets.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is a schematic illustration of a bioprocessing system including a process vessel and a plurality of filter elements.

FIG. 2 is a schematic illustration of an example valve for use in the bioprocessing system of FIG. 1;

FIGS. 3A-F are various views of an example valve for use in the bioprocessing system of FIG. 1;

FIGS. 4A-I are various views of another example valve for use in the bioprocessing system of FIG. 1; and

FIGS. 5A-5J are various vies of a further example valve for use in the bioprocessing system of FIG. 1.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more features of this disclosure. Each example of an embodiment is provided by way of explanation and is not the only way to implement these features but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed features will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. thus, it is intended that the present subject matter covers such modifications and variations as come within the scope of the appended claims and their equivalents.

Embodiments of the present disclosure relate generally to systems and methods for perfusion cell culture involving alternating fluid flows between a process vessel and one or more filtration apparatus. Fluids such as suspension cell cultures are passed through a filtration apparatus, which as an alternating tangential flow (ATF) filtration apparatus as they move back and forth between the process vessel and the filtration apparatus. As the fluids flow through the filter, they are separated into (I) a permeate flow comprising material that has passed through a membrane of the tangential flow filtration apparatus, and (II) a feed/retentate flow that has not passed through a membrane of the tangential flow filtration apparatus.

Reduction of filter permeability can be measured by the increased pressure required to push the fluid through a filter or filters. The increase of pressure or vacuum can be measured by a pressure measuring device, and can be used for an automated re-direction of flow from a fouling filter to a new one.

The disclosed system employs one or more rotary valves as in the retentate, permeate, and/or gas supply lines which result in minimal hold-up volumes in the system as during filter switching operations. The disclosed use of rotary valves enables instant switching between filters in a multi-filter arrangement. The rotary valves used as retentate and/or permeate valves switch flow to and from a first filter to a next filter in a queue of filters while closing off flow to the remaining filters so that no valuable product is lost once the flow is redirected from the first filter to the next filter.

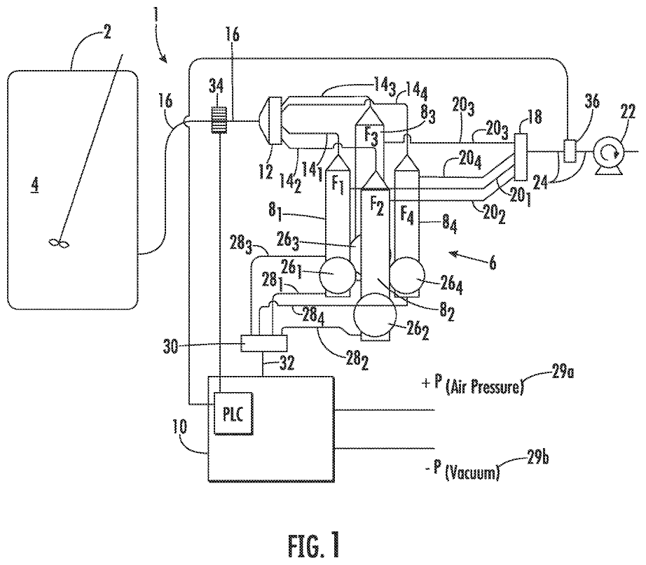

With reference to FIG. 1 an embodiment of a bioprocessing system 1 will now be described. The system 1 may include a process vessel 2 (which in one nonlimiting example embodiment is a bioreactor) containing a fluid 4, a filtration assembly 6 comprising a plurality of filter housings 8₁, 8₂, 8₃, 8₄, and a control system 10. The fluid 4 (also referred to as a feed), may contains, for example, cells, cell debris, cell metabolites including waste metabolites, expressed proteins, etc. It will be appreciated that although the description will proceed in relation to a system 1 having four filter housings, that the system can have greater or fewer numbers of filter housings (and filters) without departing from the disclosure.

The filter housings 8₁, 8₂, 8₃, 8₄ can be individually coupled to a retentate valve 12 via retentate lines 14₁, 14₂, 14₃, 14₄. The retentate valve 12 can be coupled to the process vessel 2 via combined retentate line 16. The filter housings 8₁, 8₂, 8₃, 8₄ are also individually coupled to a permeate valve 18 via permeate lines 20₁, 20₂, 20₃, 20₄. The permeate valve 18 can be coupled to a permeate pump 22 via combined permeate line 24. The permeate pump 22 can be coupled to a collection vessel (not shown) for collection and retention of the permeate received from the filter housings 8₁, 8₂, 8₃, 8₄.

The system 1 may be configured to provide alternating tangential flow (ATF) filtration of the fluid 4 in the process vessel 2. Thus, the filter housings 8₁, 8₂, 8₃, 8₄ may contain hollow fiber filter elements that can be used to separate permeate from the fluid 4 received from the process vessel 2, and the system 1 can return the fluid (retentate) to the process vessel. In the illustrated embodiment the individual filter housings 8₁, 8₂, 8₃, 8₄ may be coupled to respective individual pumps 26₁, 26₂, 26₃, 26₄, which in the illustrated embodiment are pneumatically operated diaphragm pumps, although this is not critical and other pump types (i.e., non-pneumatically actuated) can be used. The pumps 26₁, 26₂, 26₃, 26₄ are coupled to the filter housings 8₁, 8₂, 8₃, 8₄ at an end opposite the retentate lines 14₁, 14₂, 14₃, 14₄. It will be appreciated that although an ATF filtration arrangement is illustrated and described above. The ATF filtration can be achieved by a diaphragm pump but also by a variety of different methods of alternating flow through the filter. The system could also be arranged for a tangential flow filtration (TFF) without departing from the disclosure.

The pumps 26₁, 26₂, 26₃, 26₄ can be used to move the fluid 4 from the vessel 2 through the hollow fiber filter elements in the individual filter housings 8₁, 8₂, 8₃, 8₄ and into the pumps, then reversing the fluid flow from the pumps back through the hollow fiber filter elements in the filter housings to the vessel 2. In this way, an alternating tangential flow of fluid is generated through hollow fiber filter elements. As will be understood, alternating flow of retentate between pumps 26₁, 26₂, 263, 26₄ and vessel 2 is through a lumen side of the hollow fiber filter elements, while permeate passes through the walls of the hollow fiber filter elements and is directed out through the permeate lines 20₁, 20₂, 20₃, 20₄ associated with each of the filter housings 8₁, 8₂, 8₃, 8₄.

As the system 1 cycles the fluid 4 through the filtration assembly 6, the hollow fiber filter elements disposed in the filter housings 8₁, 8₂, 8₃, 8₄ remove or separate various materials (e.g., cell debris, soluble and insoluble cell metabolites and other products produced by cells including expressed proteins, viruses, virus like particles (VLPs), exosomes, lipids, DNA, or other small particles) (collectively “permeate”) from the fluid 4 and returns cells to the process vessel 2 to allow the reaction in the process vessel to continue. Removing waste metabolites allows the continued proliferation of cells within the process vessel 2, thereby allowing the cells to continue to express recombinant proteins, antibodies or other biological materials of interest.

In the non-limiting illustrated embodiment. the pumps 26₁, 26₂, 26₃, 26₄ are pneumatically actuated diaphragm pumps. It will be appreciated that other types of pumps (e.g., centrifugal levitating magnetic pumps, positive displacement pumps, peristaltic pumps, membrane pumps, and the like) can be used without departing from the spirit of the disclosure. Likewise, the permeate pump 22 can be any of a variety of pump types, including a centrifugal levitating magnetic pump, a positive displacement pump, a peristaltic pump, a membrane pump, and the like.

In the illustrated embodiment, pneumatic actuation of one or more of the pumps 26₁, 26₂, 26₃, 26₄ can be controlled by feeding a gas, such as air through reversible inlet/exhaust lines 28₁, 28₂, 28₃, 28₄ associated with each pump. The inlet/exhaust lines 28₁, 28₂, 28₃, 28₄ can be coupled to the pumps 26₁, 26₂, 26₃, 26₄ so that when the gas is passed through the lines 28₁, 28₂, 28₃, 28₄, such as from an air supply 29a, it expands a diaphragm in the pumps to move fluid in a direction toward and through the hollow fiber filter elements in the filter housings 8₁, 8₂, 8₃, 8₄. The filtered fluid is thus moved toward the process vessel 2. When the gas is drawn back through inlet/exhaust lines 28₁, 28₂, 28₃, 28₄, such as by a vacuum source 29b, the diaphragm is retracted, causing fluid from the process vessel 2 to be drawn through the hollow fiber filter elements in the filter housings 8₁, 8₂, 8₃, 8₄ into the pumps 26₁, 26₂, 26₃, 26₄.

In the illustrated embodiment, the inlet/exhaust lines 28₁, 28₂, 28₃, 28₄ are coupled to a gas valve 30, which in turn is coupled via combined gas line 32 to gas supply 29a and vacuum source 29b. Bidirectional flow control of gas through gas line 32 may be regulated by the control system 10 via an appropriate solenoid valve (not shown). This bidirectional flow control of gas can repeat in order to draw fluid back and forth from the vessel 2, through the filter assembly 6, and into the pumps 26₁, 26₂, 26₃, 26₄, in sequence to cause the aforementioned alternating flow tangentially through the hollow fiber filter elements in the filter housings 8₁, 8₂, 8₃, 8₄.

In some embodiments a flow sensor 34 is coupled to the combined retentate line 16, while a pressure sensor 36 is coupled to the combined permeate line 24. The flow sensor 34 and the pressure sensor 36 may also be coupled to the control system 10 so that the control system can use information from the sensors to control one or more aspects of the system 1. The control system 10 may also be coupled to the gas valve 30, the retentate valve 34 and the permeate valve 36 to allow the control system to control the flow of fluids through the system 1.

Advantageously the control system 10 is operable to selectively direct and redirect flow of feed 4 from the process vessel 2 to one or more of the hollow fiber filter elements in the filter housings 8₁, 8₂, 8₃, 8₄. As will be understood, during filtration operations the lumens of the filter element(s) become fouled over time as material clogs the lumens of the filter element(s). As fouling progresses, increasing levels of suction pressure are required to evacuate the permeate from the filter housing 8₁, 8₂, 8₃, 8₄. This suction pressure can be monitored by the control system 10 via pressure sensor 36 in the combined permeate line 24 and when the pressure is determined to meet or pass a predetermined value the control system 10 can switch operation from the fouled filter(s) to a clean filter.

For example, during a filtration process flow will typically be directed through fewer than all the filter housings 8₁, 8₂, 8₃, 8₄ at any one time. Thus, where flow is directed through a first one of the filter housings (e.g., 8₁) and the control system 10 determines that negative pressure in the combined permeate line 24 has reached a predetermined value as measured by the pressure senor 36 (indicating that the hollow fiber filter element in the filter housing 8₁ has reached the end of its useful life), the control system 10 can automatically adjust the positions of the gas valve 30, retentate valve 12, and permeate valve 18 so that flow from the process vessel 2 is directed to another one of the filter housings (e.g., 8₂, 8₃, 8₄). The first filter housing can thus be taken “off line” and the hollow fiber filter element in the filter housing 8₁ can be replaced without impacting continued filtration operations of the system 1.

In other embodiments, for example when the hollow fiber filter size is not sufficient to accommodate the volume of fluid 4 being filtered then two filters may be used at the same time (e.g., 8₁, 8₂). In such a case, once the control system 10 determines that pressure in the combined permeate line 24 has reached a predetermined value as measured by the pressure senor 36 (again, indicating that the hollow fiber filter elements in the filter housing 8₁, 8₂ have reached the end of their useful lives, the control system 10 will automatically adjust the positions of the gas valve 30, retentate valve 12, and permeate valve 18 so that flow from the process vessel 2 is directed to another set of the filter housings (e.g., 8₃, 8₄). The first and second filter housing 8₁, 8₂ can thus be taken “off line” and the hollow fiber filter elements in the filter housing 8₁, 8₂ can be replaced without impacting continued filtration operations of the system 1.

The control system 10 can include a microprocessor (e.g., a programmable logic controller (PLC)) including circuitry enabling the microprocessor to execute instructions to actuate the retentate valve 12, the permeate valve 18, and the gas valve 30 to direct flow of the fluid 4 between the process vessel 2 and a selected one (or subset) of the plurality of filter housings 8₁, 8₂, 8₃, 8₄, and to direct permeate flow from the selected one (or subset) of the plurality of filter housings, based on information received from the flow sensor and the pressure sensor. Non-transitory storage media/memory may be associated with the processor for storing system parameters and/or instructions for use by the processor.

The retentate, permeate, and gas valves 12, 18, 30 may be of the same design, or they may be of different designs. In some embodiments the retentate, permeate, and gas valves 12, 18, 30 are all of a design including a single inlet and multiple selectable outlets. The retentate, permeate, and gas valves 12, 18, 30 can be single use, or they may be permanent and may be provided as part of a tube set which for sterile applications is gamma or steam sterilized. In some embodiments the retentate, permeate, and gas valves 12, 18, 30 may be coupled to a linear or rotating low voltage servo/stepper actuator (not shown) which is controlled by the control system 10.

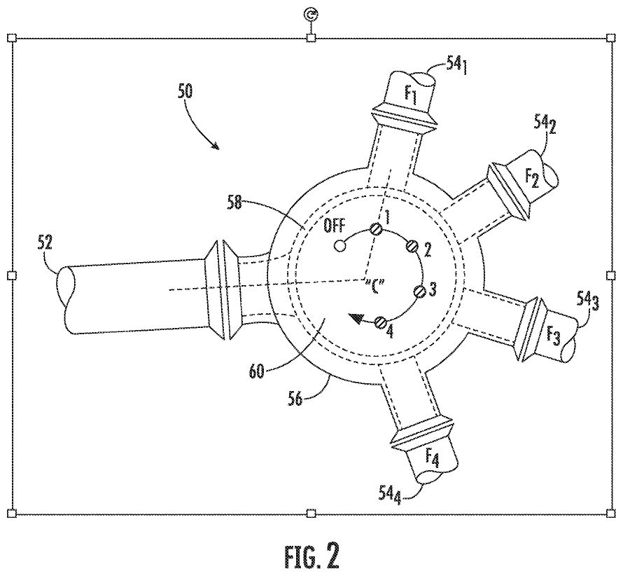

Referring now to FIG. 2, a semi-transparent view of a valve 50 for use as one or more of the retentate, permeate, and gas valves 12, 18, 30, will be described in greater detail. As mentioned, the valve 50 may include an inlet 52 and a plurality of outlets 54₁, 54₂, 54₃, 54₄.

Although the illustrated valve 50 (as well as the valves that will be subsequently described) includes four outlets, it will be appreciated that this is not limiting, and the valve 50 according to the disclosure can have any number of outlets.

The illustrated valve 50 is a rotary valve having a housing 56 that includes the inlet 52 and the plurality of outlets 54₁, 54₂, 54₃, 54₄. The housing 56 may also include an interior cavity 58 that receives a rotor 60 having a rotational axis “C”. The rotor is rotatable about the axis “C” within the housing 56 such that one or more internal fluid passageways 62 can connect the inlet 52 with one or more of the plurality of outlets 54₁, 54₂, 54₃, 54₄ as the rotor is rotated about its rotational axis. In the illustrated embodiment the internal fluid passageway 62 fluidly connects the inlet 52 with the first outlet 54₁. The rotor 60 can also have a rotational position in which the inlet is not fluidly coupled with any of the plurality of outlets 54₁, 54₂, 54₃, 54₄ (the “OFF” position shown in FIG. 2). As can be seen, as the rotor 60 is rotated to the individual rotational positions “1”, “2”, “3”, “4” the inlet 52 can be selectively aligned with tubing associated with each of the plurality of filter housings 8₁, 8₂, 8₃, 8₄. As mentioned, the rotor 60 may be coupled to a servo/stepper actuator (not shown) which is controlled by the control system 10 such that the control system can change the filter(s) through which the fluid 4 from the process vessel 2 will be directed. The valve 50 can be single use or reusable. The reusable version can be made from stainless steel with seals which are silicone or rubber appropriate for steam sterilization. The single use valve body can be manufactured from a rigid plastic and the seals are made from thermoplastic elastomers or silicone for a complete seal of the fluid or gas path. All materials should be resistant to elevated temperatures, chemicals and steam or gamma sterilization processes. The valve 50 can have different diameter inlets and outlets with different type of connection points such as luer, barb, sanitary, and the like.

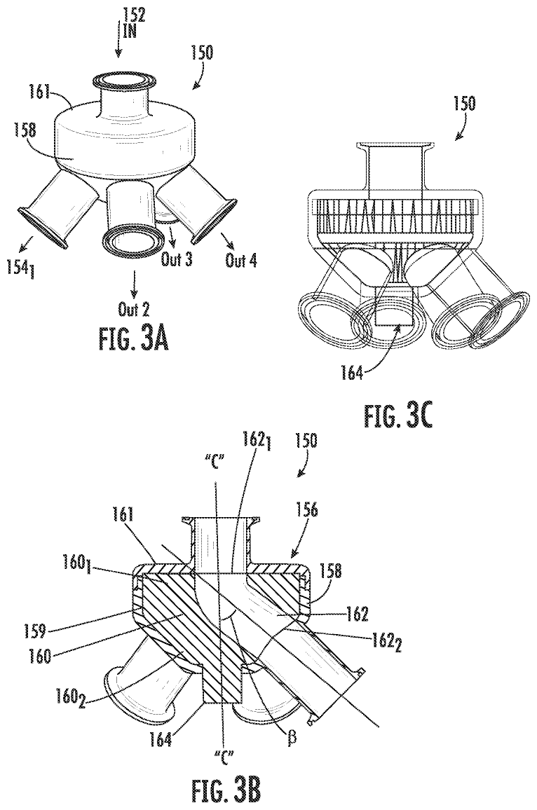

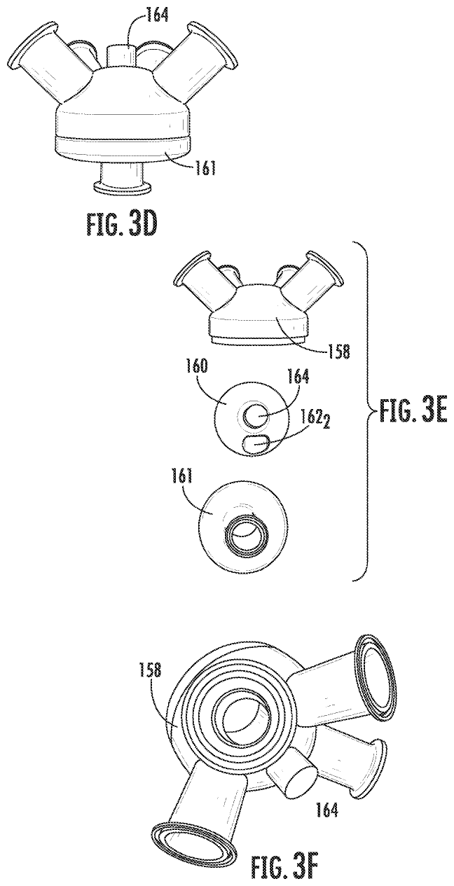

FIGS. 3A-3F illustrate an embodiment of a rotary valve 150 for use as one or more of the retentate, permeate, and gas valves 12, 18, 30. The rotary valve 150 includes an inlet 152 and a plurality of outlets 154₁, 154₂, 154₃, 154₄. When implemented as part of the system 1, the inlet 152 may be coupled directly to the combined retentate line 16 (when used as the retentate valve 12), the combined permeate line 24 (when used as the permeate valve 18), or the combined gas line 32 (when used as the gas valve 30), while the outlets 154₁, 154₂, 154₃, 154₄ can be coupled to the individual retentate lines, the individual permeate lines, or the individual gas lines.

The rotary valve 150 of this embodiment can have a housing 156 that includes a housing body 158 and a housing cap 161. The housing body 158 and housing cap 161 can together form an interior cavity 159 that receives and encloses a rotor 160. As can be seen, the rotor 160 has a cylindrical upper portion 160₁ and a cone-shaped lower portion 160₂. The interior cavity 159 has a complementary shape such that cone-shaped lower portion 1602 of the rotor sits in, and is guided by, the cone-shaped portion of the cavity.

The housing cap 161 may include the inlet 152, while the housing body 158 may include the plurality of outlets 154₁, 154₂, 154₃, 154₄. In the illustrated embodiment the inlet 152 is aligned with the rotational axis “C-C” of the rotor 160 while the outlets are oriented at an oblique angle β with respect to the axis “C-C”. The orientation of the inlet and outlets can be arranged as required by the physical placement of the valve. The spacing between location of the ports must allow shutoff between ports even under elevated pressures.

The rotor 160 is rotatable within the cavity 158 about axis “C-C” so that one or more internal fluid passageways 162 in the rotor can selectively connect the inlet 152 with one or more of the plurality of outlets 154₁, 154₂, 154₃, 154₄ as the rotor 160 is rotated about its rotational axis “C-C”. The fluid passageway 162 has an inlet end 162₁ that is aligned with the inlet 152. Both the inlet end 162₁ and inlet 152 are aligned with the axis “C-C” so that fluid received through the inlet 152 is directed into inlet end 162₁ of the fluid passageway 162 regardless of the rotational position of the rotor 160. As the rotor 160 is rotated, the outlet end 162₂ of the fluid passageway 162 can be selectively aligned with the plurality of outlets 154₁, 154₂, 154₃, 154₄ so that fluid received through the inlet 152 can be directed to one or more of the plurality of outlets 154₁, 154₂, 154₃, 154₄ based on the rotational position of the rotor 160. As can be seen, the inlet end 162₁ and outlet end 162₂ are connected by a gentle bend which facilitates laminar flow of fluid therethrough and minimizes or prevents turbulent flow. Since each of the plurality of outlets 154₁, 154₂, 154₃, 154₄ can be associated with a particular one of the plurality of filter housings 8₁, 8₂, 8₃, 8₄, connecting the inlet 152 with a particular outlet 154₁, 154₂, 154₃, 154₄ in turn connects the inlet to a selected one of the filter housings.

The housing body 158 may include an opening 159 through which an extension member 164 extends in a direction opposite to that of the inlet 152 of the housing cap 161. In the illustrated embodiment the opening 159 and the extension member 164 are aligned with the axis “C-C” so that rotation of the extension member 164 rotates the rotor 160 about the axis “C-C”. Thus arranged, the rotor 160 can be rotated to individual rotational positions such that the fluid passageway 162 aligns the inlet 152 with one or more of the plurality of outlets 154₁, 154₂, 154₃, 154₄, each of which may be coupled to tubing associated with one of the plurality of filter housings 8₁, 8₂, 8₃, 8₄. In some embodiments the rotor can also have a rotational position in which the fluid passageway 162 does not allow flow from the inlet 152 to any of the plurality of outlets 154₁, 154₂, 154₃, 154₄.

The extension member 164 can be coupled to a servo/stepper actuator (not shown) which is controlled by the control system 10 such that the control system can change the rotational position of the rotor 160 to orient fluid passageway to couple the inlet 152 to a desired one or more of the plurality of outlets 154₁, 154₂, 154₃, 154₄, each of which may be associated with one of the filter housings 8₁, 8₂, 8₃, 8₄. The servo/stepper actuator can precisely align the inlet 152 with one or more of the plurality of outlets 154₁, 154₂, 154₃, 154₄ at a desired speed. Speed of transition from a filter to a filter can be important for the more sensitive cell cultures.

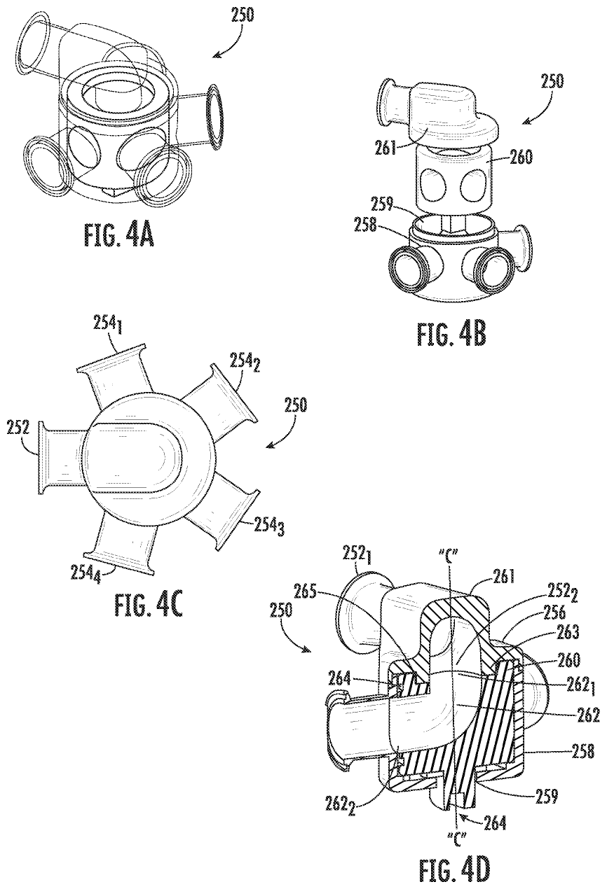

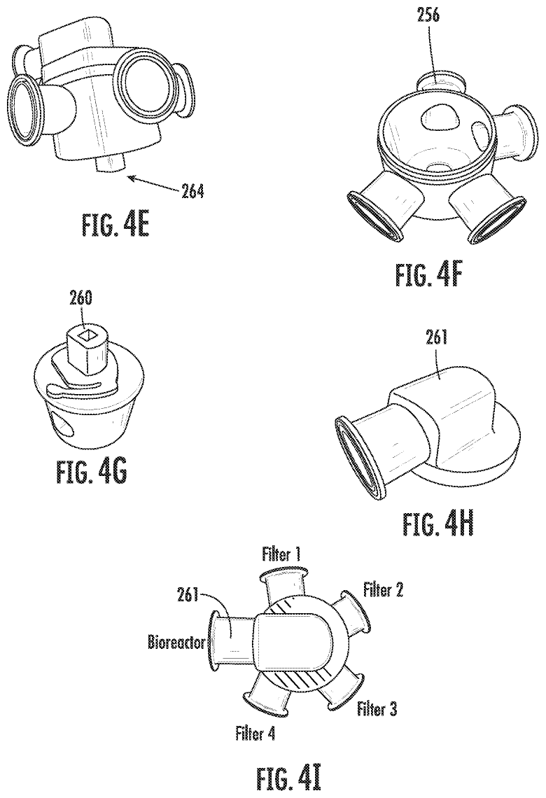

FIGS. 4A-4F illustrate an embodiment of a rotary valve 250 for use as one or more of the retentate, permeate, and gas valves 12, 18, 30. The rotary valve has an inlet 252 and a plurality of outlets 254₁, 254₂, 254₃, 254₄. When implemented as part of the system 1, the inlet 252 may be coupled directly to the combined retentate line 16 (when used as the retentate valve 12), the combined permeate line 24 (when used as the permeate valve 18), or the combined gas line 32 (when used as the gas valve 30), while the outlets 254₁, 254₂, 254₃, 254₄ can be coupled to the individual retentate lines, the individual permeate lines, or the individual gas lines.

The rotary valve 250 of this embodiment can have a housing 256 that includes a housing body 258 and a housing cap 261. The housing body 258 and housing cap 261 can together form an interior cavity 259 that receives and encloses a rotor 260. As can be seen, the rotor 260 is generally cylindrical, and the interior cavity 259 of the housing body 258 has a complementary shape such that the rotor 260 sits in, and is guided by, the cylindrical interior cavity 259. The housing cap 261 includes a cylindrical ridge 263 that fits within a cylindrical recess 265 in the rotor 260 to maintain a desired orientation of the rotor 260 as it rotates within he housing body 258.

The housing cap 261 includes the inlet 252, while the housing body 258 includes the plurality of outlets 254₁, 254₂, 254₃, 254₄. In the illustrated embodiment the inlet 252 is aligned with the rotational axis “C-C” of the rotor 260, while the outlets are oriented orthogonal to the axis “C-C”. As can be seen, the inlet 252 and the plurality of outlets 254₁, 254₂, 254₃, 254₄ are oriented radially outward from the rotational axis “C-C” of the rotor 260 and are evenly spaced apart from each other.

The rotor 260 is rotatable within the cavity 258 about axis “C-C” so that an internal fluid passageway 262 in the rotor can selectively connect the inlet 252 with one or more of the plurality of outlets 254₁, 254₂, 254₃, 254₄ as the rotor 260 is rotated about its rotational axis “C-C”. The fluid passageway 262 has an inlet end 262₁ that is in fluid communication with the inlet 252. The inlet end 262₁ of the fluid passageway is aligned with the axis “C-C”. In the illustrated embodiment an outer end 252₁ of the valve inlet 252 is oriented orthogonal to the axis “C-C”, thus allowing the inlet 252 of the valve 250 to couple to tubing that is oriented orthogonal to the axis “C-C”. Internal to the housing cap 261 a bend portion orients the inner end 252₂ of the inlet 252 to align with the inlet end 262₁ of the fluid passageway so that fluid received through the valve inlet 252 is directed into inlet end 262₁ of the fluid passageway 262 regardless of the rotational position of the rotor 260. As can be seen, the bend portion between the inlet and outlet ends 262₁, 262₂ facilitates laminar flow of fluid therethrough and minimizes or prevents turbulent flow. As the rotor 260 is rotated, the outlet end 262₂ of the fluid passageway 262 can be selectively aligned with one of the plurality of outlets 254₁, 254₂, 254₃, 254₄ so that fluid received through the inlet 252 can be directed to one or more of the plurality of outlets 254₁, 254₂, 254₃, 254₄ based on the rotational position of the rotor 260. In the illustrated embodiment, the outlet end 262₂ can be sealed against the selected one of the plurality of outlets 254₁, 254₂, 254₃, 254₄ by an O-ring (not shown) receivable in a circular groove 264 disposed in the rotor 260 in a region surrounding the outlet end 262₂ of the fluid passageway 262. Since each of the plurality of outlets 254₁, 254₂, 254₃, 254₄ can be associated with a particular one of the plurality of filter housings 8₁, 8₂, 8₃, 8₄, connecting the inlet 252 with a particular outlet 254₁, 254₂, 254₃, 254₄ in turn connects the inlet to a selected one of the filter housings.

The housing body 258 may include an opening 259 through which an extension member 264 extends in a direction opposite to that of the inlet 252 of the housing cap 261. In the illustrated embodiment the opening 259 and the extension member 264 are aligned with the axis “C-C” so that rotation of the extension member 264 rotates the rotor 260 about the axis “C-C”. Thus arranged, the rotor 260 can be rotated to individual rotational positions such that the fluid passageway 262 aligns the inlet 252 with one or more of the plurality of outlets 254₁, 254₂, 254₃, 254₄, each of which may be coupled to tubing associated with one of the plurality of filter housings 8₁, 8₂, 8₃, 8₄. In some embodiments the rotor can also have a rotational position in which the fluid passageway 262 does not allow flow from the inlet 252 to any of the plurality of outlets 154₁, 154₂, 154₃, 154₄.

All of the rotary valves disclosed herein can be configured with a single inlet and a single outlet. In some embodiments where the filtration area of a single filter is not sufficient for a specific application, the user may select an option in which two active filters receive fluid at the same time to double the filtration area. For example, if actively-working filters 1 and 2 are fouling, the PLC can command a flow redirection to filters 3 and 4. The disclosed valves are modular and can accept a rotor with one inlet and one outlet, or one inlet and two outlets to enable flow to be directed to two filters at the same time.

The extension member 264 can be coupled to a servo/stepper actuator (not shown) which is controlled by the control system 10 such that the control system can change the rotational position of the rotor 160 to orient fluid passageway to couple the inlet 252 to a desired one or more of the plurality of outlets 254₁, 254₂, 254₃, 254₄, each of which may be associated with one of the filter housings 8₁, 8₂, 8₃, 8₄. The actuator can be preprogramed for a specific rotational movement based on the location of the plurality of outlets 254₁, 254₂, 254₃, 254₄. Feedback from the actuator encoder can guarantee precise alignment of ports and speed to get from a location to a location.

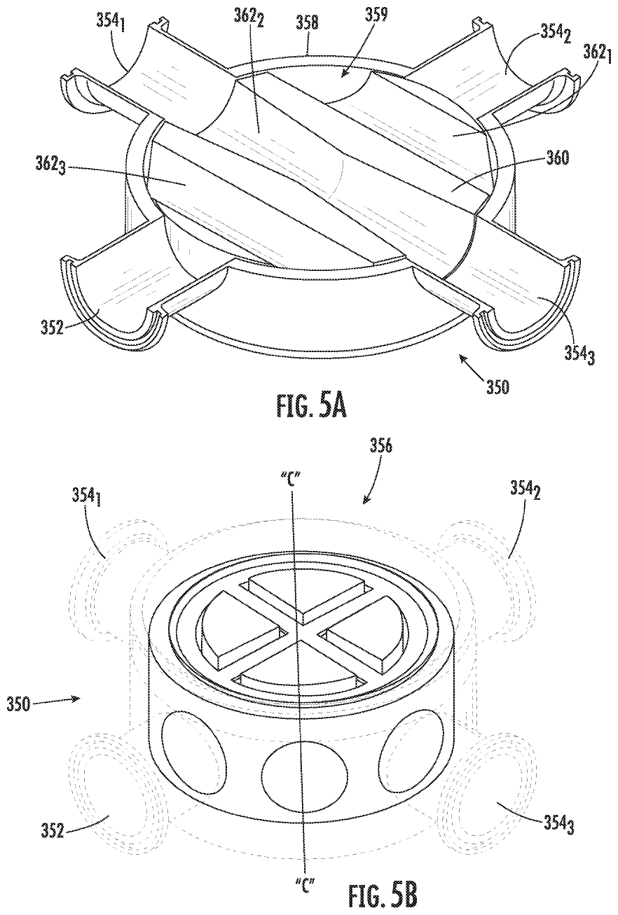

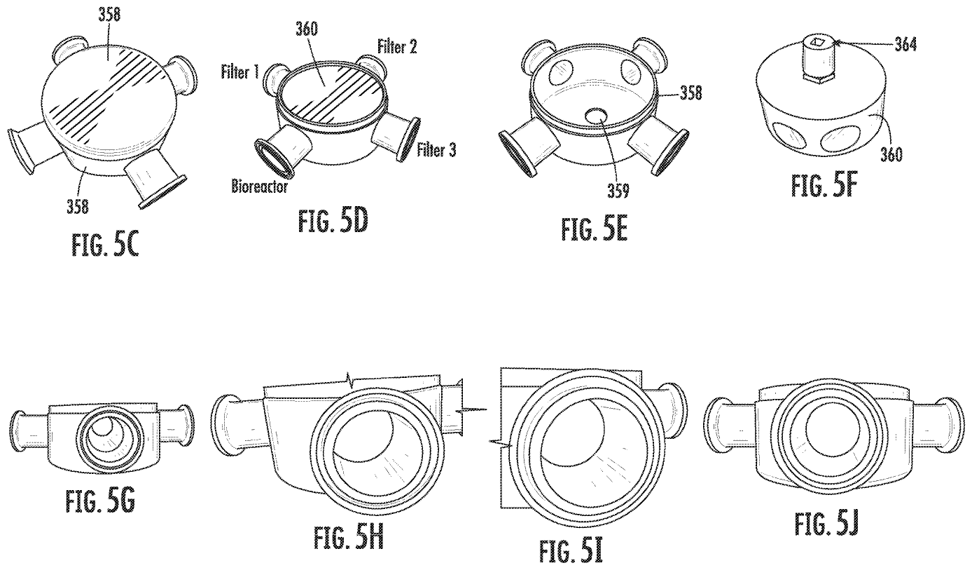

FIGS. 5A-5J illustrate an embodiment of a rotary valve 350 for use as one or more of the retentate, permeate, and gas valves 12, 18, 30. The rotary valve 350 includes an inlet 352 and a plurality of outlets 354₁, 354₂, 354₃. When implemented as part of an embodiment of the system 1 that includes three individual filters 8₁, 8₂, 8₃, the inlet 352 may be coupled directly to the combined retentate line 16 (when used as the retentate valve 12), the combined permeate line 24 (when used as the permeate valve 18), or the combined gas line 32 (when used as the gas valve 30), while the outlets 354₁, 354₂, 354₃ can be coupled to the individual retentate lines, the individual permeate lines, or the individual gas lines.

The rotary valve 350 of this embodiment can have a housing 356 that includes a housing body 358 and a housing cap 361. The housing body 358 and housing cap 361 can together form an interior cavity 359 that receives and encloses a rotor 360. As can be seen, the rotor 360 is generally cylindrical, and the interior cavity 359 of the housing body 358 has a complementary shape such that the rotor 360 sits in, and is guided by, the interior cavity 359.

The housing cap 361 includes the inlet 352, while the housing body 358 includes the plurality of outlets 354₁, 354₂, 354₃. In the illustrated embodiment the inlet 352 and the plurality of outlets 354₁, 354₂, 354₃ are oriented orthogonal to the axis “C-C”. The 352 and the plurality of outlets 354₁, 354₂, 354₃ are also oriented radially outward from the rotational axis “C-C” of the rotor 360 and are evenly spaced apart from each other.

The rotor 360 of this embodiment includes a plurality of internal fluid passageways 362₁, 362₂, 362₃ which are oriented generally parallel to each other. The rotor 360 is rotatable within the cavity 358 about axis “C-C” so that one or more internal fluid passageways 362₁, 362₂, 362₃ in the rotor can selectively connect the inlet 352 with one or more of the plurality of outlets 354₁, 354₂, 354₃ as the rotor 360 is rotated about its rotational axis “C-C”. Since each of the plurality of outlets 354₁, 354₂, 354₃ can be associated with a particular one of the plurality of filter housings 8₁, 8₂, 8₃, connecting the inlet 352 with a particular outlet 354₁, 354₂, 354₃ in turn connects the inlet to a selected one of the filter housings.

The housing body 358 may include an opening 359 (FIG. 5E) through which an extension member 364 (FIG. 5F) of the rotor 360 extends. In the illustrated embodiment the opening 359 and the extension member 334 are aligned with the axis “C-C” so that rotation of the extension member 364 rotates the rotor 360 about the axis “C-C”. Thus arranged, the rotor 360 can be rotated to individual rotational positions (FIGS. 5G-FI) such that the fluid passageways 362₁, 362₂, 362₃ align the inlet 352 with one or more of the plurality of outlets 354₁, 354₂, 354₃, each of which may be coupled to tubing associated with one of the plurality of filter housings 8₁, 8₂, 8₃. In FIG. 5G a first rotational position of the rotor 360 aligns passageway 362₂ such that fluid can pass between the inlet 352 and outlet 354₂. In FIG. 5H a second rotational position of the rotor 360 aligns passageway 362₃ such that fluid can pass between the inlet 352 and outlet 354₁. In FIG. 5I a rotational position of the rotor 360 aligns passageway 362₁ such that fluid can pass between the inlet 352 and outlet 354₃. In some embodiments the rotor 360 can also have a rotational position (FIG. 5J) in which the fluid passageway 332 does not allow flow from the inlet 352 to any of the plurality of outlets 354₁, 354₂, 354₃. As previously mentioned, the rotary valve 350 can be modular so that it can be used with multiple different rotor configurations. For example, the rotary valve 350 can incorporate a rotor configured to couple between a single inlet and a single outlet (i.e., where only a single filter is used at any one time). The rotary valve 350 can also incorporate a rotor configured to couple between a single inlet and multiple outlets (i.e., where multiple filters are used at the same time).

The extension member 364 can be coupled to a servo/stepper actuator (not shown) which is controlled by the control system 10 such that the control system can change the rotational position of the rotor 360 to orient passageways 362₁, 362₂, 362₃ to couple the inlet 352 to a desired one or more of the plurality of outlets 354₁, 354₂, 354₃, each of which may be associated with one of the filter housings 8₁, 8₂, 8₃. In some embodiments multiple valves 350 can be connected in series. Such a series connection arrangement provides a variety of options to facilitate connectivity of filters in a multi-filter system.

While the disclosure herein disclosed has been described by means of specific embodiments and applications thereof, numerous modifications and variations could be made thereto by those skilled in the art without departing from the scope of the disclosure set forth in the claims.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

Various features, aspects, or the like of a vessel or process system may be used independently of, or in combination, with each other. It will be appreciated that a vessel and/or system as disclosed herein may be embodied in many different forms and should not be construed as being limited to the illustrated embodiments of the figures, such as described herein. Rather, these embodiments are provided so that this disclosure will convey certain aspects of a vessel and/or process system formed in accordance with various principles of the present disclosure to those skilled in the art.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein. Only one of the various features may be present in a vessel or system formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

1. A bioprocessing system, comprising:
a process vessel;
a filtration assembly including a plurality of filter housings;
a feed pump coupled to the plurality of filter housings for moving fluid between the process vessel and the filtration assembly;
a retentate valve coupled within a retentate line between the process vessel and the plurality of filter housings, the retentate valve including a fluid passageway that is controlled to selectively direct movement of the fluid to at least one of the plurality of filter housings;
a permeate valve coupled within a permeate line between the plurality of filter housings and a permeate pump;
a pressure sensor disposed in the permeate line; and
a controller coupled to the pressure sensor, the retentate valve, and the permeate valve, the controller executing instructions for actuating the retentate valve and the permeate valve to direct flow between the process vessel and a selected one of the plurality of filter housings, and to direct permeate flow from the selected one of the plurality of filter housings to the permeate pump, based on information received from the pressure sensor.

2. The system of claim 1, further comprising a pneumatic valve coupled between a source of air pressure and a plurality of pneumatically actuated pumps associated with respective ones of the plurality of filter housings.

3. The system of claim 2, a flow sensor disposed in the retentate line for monitoring a flowrate of the feed to and from the process vessel.

4. The system of claim 1, wherein the retentate valve is a rotary retentate valve having a rotor with a fluid passageway, the rotor being rotatable to orient the fluid passageway to fluidly connect an inlet of the rotary retentate valve with a selected outlet of the rotary retentate valve.

5. The system of claim 4, wherein the rotary retentate valve comprises a plurality of outlets, and wherein each of the plurality of outlets is fluidly coupled to a respective one of the plurality of filter housings.

6. The system of claim 5, wherein the fluid passageway comprises a plurality of fluid passageways, and the selected outlet comprises a plurality of selected outlets, and wherein the rotor is rotatable to orient the plurality of fluid passageways to fluidly connect the inlet of the rotary retentate valve with the selected plurality of outlets.

7. The system of claim 1, wherein the permeate valve is a rotary permeate valve having a rotor with a fluid passageway, the rotor being rotatable to orient the fluid passageway to fluidly connect an inlet of the rotary permeate valve with a selected outlet of the rotary permeate valve.

8. The system of claim 7, wherein the inlet of the rotary permeate valve is fluidly coupled to the permeate line.

9. The system of claim 1, wherein the retentate valve is a rotary retentate valve having a housing and a rotor disposed within the housing, the rotor including a cylindrical portion and a conical portion, the conical portion seated within a corresponding conical portion of the housing and disposed for rotational movement about a rotational axis therein.

10. The system of claim 1, wherein the retentate valve is a rotary retentate valve having a housing and a cylindrical rotor disposed within the housing, the cylindrical rotor including a plurality of fluid passageways for selectively fluidly coupling an inlet of the rotary retentate valve to a selected one of a plurality of outlets of the rotary retentate valve.

11. The system of claim 9, wherein the rotary retentate valve includes an extension disposed through the housing, the extension coupled to an actuator controllable by the controller to change a rotational position of the rotor to orient at least one of a plurality of fluid passageways in the rotor to couple an inlet of the rotary retentate valve to a desired one of a plurality of outlets of the rotary retentate valve, wherein each of plurality of outlets is fluidly coupled to a respective one of the plurality of filter housings.

12. A rotary valve for use in a bioprocessing system, comprising:
a housing comprising a housing body and a housing cap, the housing body comprising a plurality of fluid outlets; and
a rotor disposed within the housing body and having a fluid passageway, the rotor being rotatable within the housing body to fluidly connect a fluid inlet to a selected one of said plurality of fluid outlets.

13. The valve of claim 12, the rotor further including an extension member disposed through the housing and couplable to an actuator for rotating the rotor.

14. The valve of claim 13, wherein the fluid passageway comprises a plurality of fluid passageways.

15. The valve of claim 14, wherein the plurality of fluid passageways comprise three parallel fluid passageways.

16. The valve of claim 14, wherein the plurality of fluid passageways each includes a bend having a geometry configured to facilitate laminar flow through the valve.

17. The valve of claim 12, wherein the inlet is disposed in the housing cap, the inlet being fluidly coupled to an inlet portion of the fluid passageway.

18. The valve of claim 12, wherein the inlet is oriented to direct flow into the fluid passageway along a rotational axis of the rotor.

19. The valve of claim 12, wherein the inlet is disposed in the housing body and is oriented orthogonal to a rotational axis of the rotor.

20. The valve of claim 12, wherein the fluid passageway comprises a plurality of fluid passageways configured such that the rotor is rotatable to a first predetermined position in which the inlet is fluidly coupled to a first selected pair of fluid outlets of the plurality of fluid outlets.

21. (canceled)
